(19) United States
(12) Patent Application Publication
(10) Pub. No.: US 2023/0233221 A1
(43) Pub. Date: Jul. 27, 2023

(54) Aspiration Catheter with an Adjustable Tip for the Intracranial Circulation

(71) Applicant: The University of Toledo, Toledo, OH (US)

(72) Inventors: Mouhammad A. Jumaa, Toledo, OH (US); Syed F. Zaidi, Toledo, OH (US); Julie Shawver, Toledo, OH (US)

(73) Assignee: The University of Toledo, Toledo, OH (US)

(21) Appl. No.: 18/101,812

(22) Filed: Jan. 26, 2023

Related U.S. Application Data
(60) Provisional application No. 63/303,605, filed on Jan. 27, 2022.

(51) Int. Cl. A61B 17/221; A61M 25/01; A61M 25/00

(52) U.S. Cl. CPC A61B 17/221; A61M 25/0108; A61M 25/0045; A61M 25/0147; A61B 2017/22079

(57) ABSTRACT

An aspiration catheter useful for intracranial thrombus removal is described, along with methods of using the same. The aspiration catheter includes an adjustable tip having sides that are adjustable by an operator through the use of pull wires that extend along, and protrude from, the aspiration catheter.

### RELATED APPLICATIONS

This application claims priority to U.S. Provisional Application No. 63/303,605 filed under 35 U.S.C. § 111(b) on Jan. 27, 2022, the entire disclosure of which is incorporated herein by reference for all purposes.

### STATEMENT REGARDING FEDERALLY SPONSORED RESEARCH

This invention was made with no government support. The government has no rights in this invention.

### BACKGROUND

Acute ischemic stroke is the sudden blockage of adequate blood flow to a section of the brain, usually caused by thrombus or other emboli lodging or forming in one of the blood vessels supplying the brain. If the blockage is not quickly resolved, the ischemia may lead to permanent neurologic deficit or death. The intracranial use of aspiration catheters is becoming part of the standard of care for stroke patients, to suction a clot out of a blood vessel and restore intracranial circulation and brain function early on. Thrombus aspiration therapy is achieved by inserting a small tube called a cather from the leg or arm to reach a desired location in the blood vessel and sucking and removing debris such as thrombus and atheromas released into the blood vessel. However, the path to the brain is very tortuous, and fragments of clots that break off can go into smaller blood vessels, which is why the first pass with an aspiration catheter is so important. There is a need in the art for new and improved intracranial catheters.

### SUMMARY

Provided is an aspiration catheter comprising an elongated, flexible tube defining a lumen and extending from a proximal end to a distal end; an adjustable tip at the distal end; a first pull wire protruding from the proximal end to create a first pull wire protrusion and extending along the tube to the adjustable tip, wherein the first pull wire is connected to a first side of the adjustable tip; and a second pull wire protruding from the proximal end to create a second pull wire protrusion and extending along the tube to the adjustable tip, wherein the second pull wire is connected to a second side of the adjustable tip; wherein tension on the first pull wire causes the first side of the adjustable tip to recede toward the proximal end, and tension on the second pull wire causes the second side of the adjustable tip to recede toward the proximal end. This creates an interactive mechanism to change the tip of the catheter from round to beveled in two orientations and back to round when the pull wires are relaxed.

In certain embodiments, the tension on the first pull wire is from pulling the first pull wire protrusion, and the tension on the second pull wire is from pulling the second pull wire protrusion.

In certain embodiments, the adjustable tip can be adjusted through the pull wires to form an angled or beveled tip.

In certain embodiments, the adjustable tip comprises a first region of nitinol coil with a braid, and a second region of nitinoil coil with a braid, wherein the braid in the second region is less dense than the braid in the first region, and wherein the second region is closer to the distal end than the first region.

In certain embodiments, the adjustable tip comprises a first region of nitinol coil with a braid, and a second region of nitinoil coil with no braid, wherein the second region is closer to the distal end than the first region.

In certain embodiments, the aspiration catheter further comprises a suction device connected to the tube at the proximal end and configured to provide suction to the lumen.

In certain embodiments, the tube is at least 135 cm in length from the proximal end to the distal end.

In certain embodiments, the aspiration catheter further comprises at least one radiopaque marker on the adjustable tip. In certain embodiments, the aspiration catheter further comprises two or more radiopaque markers on the adjustable tip.

In certain embodiments, the aspiration catheter further comprises a third pull wire on a third side of the adjustable tip, and a fourth pull wire on a fourth side of the adjustable tip. In particular embodiments, the aspiration catheter further comprises a first radiopaque marker near the first pull wire, a second radiopaque marker near the second pull wire, a third radiopaque marker near the third pull wire, and a fourth radiopaque marker near the fourth pull wire.

In certain embodiments, the elongated, flexible tube is fabricated from a memory alloy, a superelastic material, an elastic material, or a combination thereof. In certain embodiments, the elongated, flexible tube comprises titanium, vanadium, aluminum, nickel, tantalum, zirconium, chromium, silver, gold, silicon, magnesium, niobium, scandium, platinum, cobalt, palladium, manganese, molybdenum, or alloys thereof. In certain embodiments, the elongated, flexible tube comprises zirconium-titanium-tanalum alloys, cobalt-chromium-molybdenum alloys, nitinol, or stainless steel.

In certain embodiments, the elongated, flexible tube comprises a polyamide elastomer, polyurethane, polyester elastomer, polyethylene, or polyether ether ketone.

In certain embodiments, the elongated, flexible tube includes a hydrophilic coating on an outer surface thereof.

Further provided is a method of removing a thrombus, the method comprising navigating the elongated, flexible tube of the aspiration catheter described herein through a blood vessel to a thrombus; applying tension on the first pull wire or the second pull wire to configure the adjustable tip in a manner desired for capturing the thrombus; and applying suction to the elongated, flexible tube to remove the thrombus. In certain embodiments, the thrombus is located in a brain.

In certain embodiments, the adjustable tip is configured to create a beveled tip. In particular embodiments, the method further comprises releasing the tension on the first pull wire and/or second pull wire so as to configure the adjustable tip to create a non-beveled tip.

In certain embodiments, the aspiration catheter further comprises a third pull wire and a fourth pull wire; wherein the third pull wire protrudes from the proximal end to create a third pull wire protrusion and extends along the tube to a third side of the adjustable tip, and the fourth pull wire protrudes from the proximal end to create a fourth pull wire protrusion and extends along the tube to a fourth side of the adjustable tip; and wherein tension on the third pull wire from pulling the third pull wire protrusion causes the third side of the adjustable tip to recede toward the proximal end, and tension on the fourth pull wire from pulling the fourth pull wire protrusion causes the fourth side of the adjustable tip to recede toward the proximal end.

In certain embodiments, the adjustable tip comprises two or more radiopaque markers configured to allow a user to visualize the manner in which the adjustable tip is configured. In particular embodiments, the adjustable tip comprises a first radiopaque marker on the first side, a second radiopaque marker on the second side, a third radiopaque marker on the third side, and a fourth radiopaque marker on the fourth side.

### BRIEF DESCRIPTION OF THE DRAWINGS

FIG. 1: Perspective view of an aspiration catheter with an adjustable tip in accordance with the present disclosure

FIG. 2: View of the adjustable tip of the aspiration catheter depicted in FIG. 1.

FIGS. 3A-3B: Perspective views of adjustable tip angles created by the wire pull mechanism. FIG. 3A shows the second side of the adjustable tip receded, and FIG. 3B shows the first side of the adjustable tip receded.

FIG. 4: View of the distal end of an aspiration catheter in accordance with the present disclosure, with four pull wires attached to the adjustable tip.

FIG. 5: Perspective view of an aspiration catheter in accordance with the present disclosure, with an adjustable tip with a pull wire.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided is an aspiration catheter with an adjustable tip for improved maneuvering and clot aspiration of the intracranial circulation. Whereas some known aspiration catheters have fixed beveled tips in order to increase suction, it has been found that having a fixed beveled tip on an aspiration catheter may cause problems with tracking the catheter to the brain, thus making the catheter less effective for intracranial use. The present disclosure provides a more effective solution for the intracranial use of aspiration catheters by providing an active adjustable tip that can be made into a beveled tip, or can be kept unbeveled or otherwise returned to a non-beveled configuration, at the operator's discretion in real time. This provides the benefits of having a beveled tip, such as improved suction and flexibility, while improving the ability of the tip to be tracked and making the catheter easier to navigate, thereby improving first pass efficiency and alleviating concerns of accidental punctures.

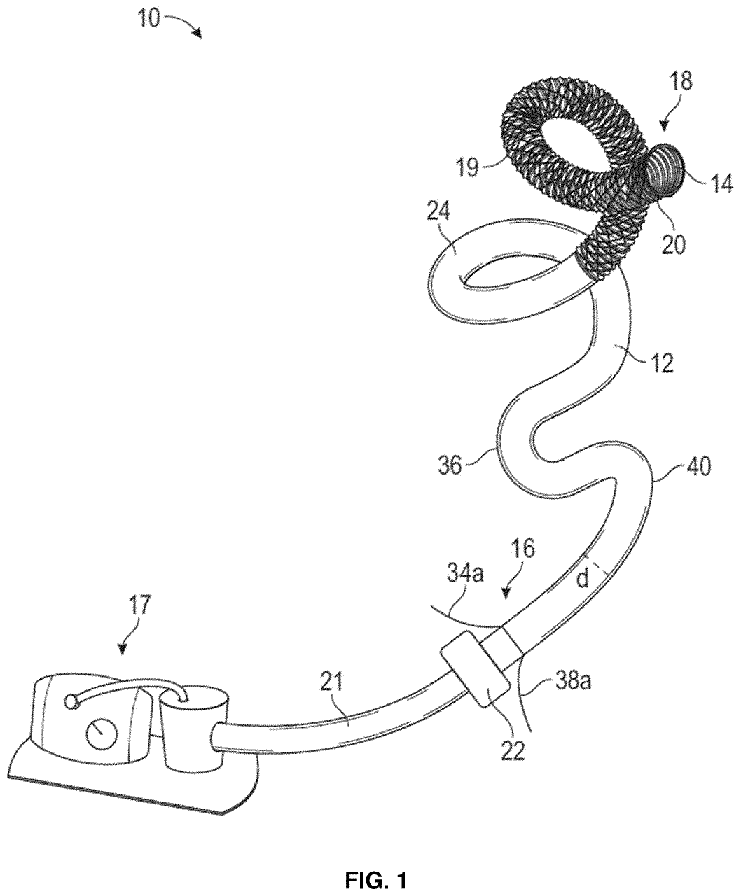

Referring now to FIG. 1, depicted is an aspiration catheter 10 in accordance with the present disclosure. The aspiration catheter 10 includes an elongated, flexible tube 12 that defines a lumen 14 therein. The lumen 14 is configured to receive fluids or thombi. The tube 12 extends from a proximal end 16 of the aspiration catheter 10 to a distal end 18 of the aspiration catheter 10. The tube 12 is made of sufficient flexibility and has a sufficiently small diameter to be inserted into, and travel through, blood vessels. The tube 12 may have any suitable length for the desired application. In one non-limiting example, the tube 12 has a length of at least about 115 cm, making it suitable for intracranial use.

Referring still to FIG. 1, the aspiration catheter 10 includes an adjustable tip 20 at the distal end 18. At the proximal end 16, the aspiration catheter 10 includes a base 22 configured to be connected to a suction device 17 to provide suction to the lumen 14. The tube 12 is configured to withstand −29 inHg vacuum. The outer diameter d of the tube 12 may be, for example, in the range of from about 0.066 inches (5 Fr) to about 0.092 inches (7 Fr). However, other diameters d of the tube 12 are possible and encompassed within the scope of the present disclosure.

Referring still to FIG. 1, the outer surface 24 of the tube 12 may include a hydrophilic coating. Suitable coatings include, but are not limited to, polymers that provide anticoagulant, anti- thrombotic, thrombolytic, fibrinoytic, or antiproliferative properties, and that preferably resist protein deposition. Non-limiting examples include polyvinylpyrrolidone, polyethylene glycol (PEG), polyethylene oxide (PEO), polyvinyl pyrrolidone (PVP), polyvinyl alcohol (PVA), and phosphoryl choline (PC). The hydrophilic coating may further include degradable polymers that release bioactive agents, such as, for example, nitric oxide releasing polymers or polyaspirin.

Referring still to FIG. 1, the tube 12 may be fabricated out of biocompatible materials, such as shape memory alloys, superelastic materials, or elastic materials, including, but not limited to, titanium, vanadium, aluminum, nickel, tantalum, zirconium, chromium, silver, gold, silicon, magnesium, niobium, scandium, platinum, cobalt, palladium, manganese, molybdenum, and alloys thereof, such as zirconium-titanium-tanalum alloys, cobalt-chromium-molybdenum alloys, nitinol, and stainless steel. Some or all of the tube 12 may include braided nitinol 19. The tube may also be made from a flexible plastic material such as, but not limited to, polyamide elastomer, polyurethane, polyester elastomer, polyethylene, or polyether ether ketone.

The aspiration catheter may include multiple regions with varying flexibility. For example, referring still to FIG. 1, the aspiration catheter 10 may include one or more segments or regions having braided nitinol 19 while other segments or regions do not include such braiding of material.

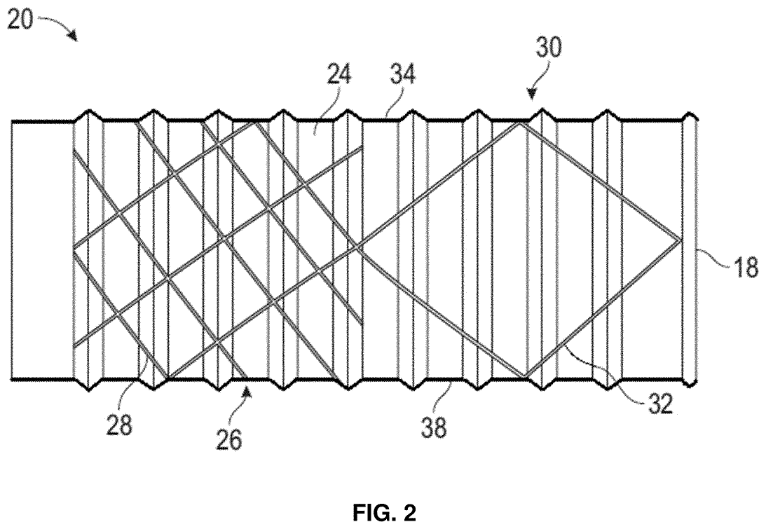

The adjustable tip 20 may include multiple regions with varying rigidity, made of different materials or of the same material having multiple layers. For example, referring now to FIG. 2, the adjustable tip 20 may include a first region 26 with a first nitinoil coil braid 28 and a second region 30 with a second nitinol coil braid 32, where the first region 26 has a more dense braid 28 than the braid 32 of the second region 30, and where the second region 30 is more distal (i.e., closer to the distal end 18) than the first region 26. In some embodiments, the second region 30 is immediately adjacent to the distal end 18 of the tube 12. The allows for the region nearest the distal end 18 (including all or portions of the adjustable tip 20) to be more flexible than other regions of the tube 12. In other embodiments, the second region 30 may include nitinol coil with no braid. In either case, this allows for the second region 30 to be more flexible than the first region 26. This is advantageous in configuring the adjustable tip 20 to capture a thrombus.

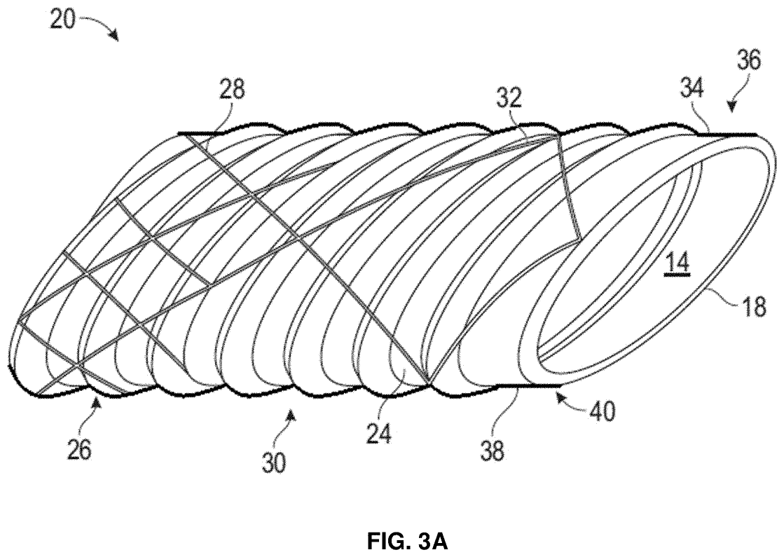

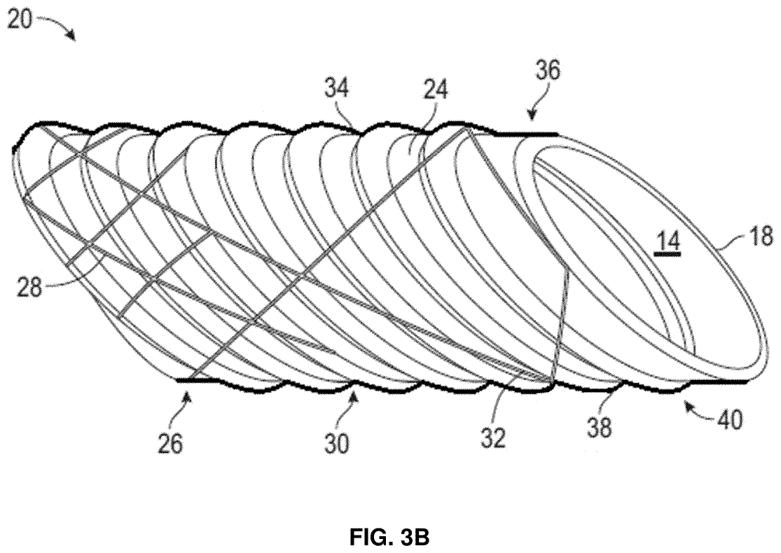

Referring to FIGS. 1-3, a first pull wire 34 is connected to the adjustable tip 20 at a first side 36 of the tube 12 (which is also the first side 36 of the adjustable tip 20) and extends along the length of the tube 12, protruding from the proximal end 16 to create a first pull wire protrusion 34a. The first pull wire 34 may be held to the tube 12 along the length from the proximal end 16 to the distal end 18 by being interwoven into the nitinol braiding along the outer surface 24 of the tube 12. In other embodiments, the first pull wire 34 may be attached to the outer surface 24 of the tube 12 at any number of points along the length from the proximal end 16 to the distal end 18 with suitable fasteners. In other embodiments, the first pull wire 34 may be housed within a first sleeve that runs along the first side 36 of the outer surface 24 of the tube 12. A second pull wire 38 is connected to the adjustable tip 20 at a second side 40 of the tube 12 (which is also the second side 40 of the adjustable tip 20) and extends along the length of the tube 12, protruding from the proximal end 16 to create a second pull wire protrusion 38a. The second pull wire 38 may be held to the tube 12 along the length from the proximal end 16 to the distal end 18 by being interwoven into the nitinol braiding along the outer surface 24 of the tube 12. In other embodiments, the second pull wire 38 may be attached to the outer surface 24 of the tube 12 at any number of points along the length from the proximal end 16 to the distal end 18 with suitable fasteners. In other embodiments, the second pull wire 38 may be housed within a second sleeve that runs along the second side 40 of the outer surface 24 of the tube 12. Tension on the second pull wire 38, or a force on the second pull wire 38 in the direction away from the distal end 18, such as from pulling on the second pull wire protrusion 38a, causes the second side 40 of the adjustable tip 20 to recede toward the proximal end 16, as seen in FIG. 3A. Tension on the first pull wire 34, or a force on the first pull wire 34 in the direction away from the distal end 18, such as from pulling on the first pull wire protrusion 34a, causes the first side 36 of the adjustable tip 20 to recede toward the proximal end 16, as seen in FIG. 3B. Thus, together, the first pull wire 34 and the second pull wire 38 can be utilized to adjust an angle or configuration of the adjustable tip 20, such as by pulling on the first pull wire protrusion 34a and/or the second pull wire protrusion 38a.

When the tension or force on the first pull wire 34 is released, the first side 36 returns to its original shape and configuration. Similarly, when the tension or force on the second pull wire 38 is released, the second side 40 returns to its original shape and configuration. This is made possible by the shape memory nature, or the stiffness and/or rigidity, of the material the tube 12 is fabricated out of. Accordingly, the adjustable tip 20 may be adjusted from a beveled tip (as depicted in FIGS. 3A-3B) into a non-beveled tip (as depicted in FIG. 2), for instance by a user releasing the first pull wire protrusion 34a and the second pull wire protrusion 38a.

Each of the pull wires 34, 38 may be fabricated out of a metal, alloy, or polymer. Non-limiting example materials include the same materials that may be used to fabricate the elongated, flexible tube 12, namely: titanium, vanadium, aluminum, nickel, tantalum, zirconium, chromium, silver, gold, silicon, magnesium, niobium, scandium, platinum, cobalt, palladium, manganese, molybdenum, and alloys thereof, such as zirconium-titanium-tanalum alloys, cobalt-chromium-molybdenum alloys, nitinol, stainless steel, a polyamide elastomer, polyurethane, polyester elastomer, polyethylene, polyether ether ketone, or a combination thereof. The composition of the pull wires 34, 38 is not limited, so long as the pull wires 34, 38 are capable of adjusting the adjustable tip 20 in the manner described herein.

Referring now to FIGS. 3A-3B, the adjustable tip 20 is capable of being adjusted into a beveled tip, where the first side 36 or the second side 40 extends at an angle beyond the other of the first or second side 36, 40, by actuation of the first pull wire 34 and/or second pull wire 38. FIG. 3B depicts the first side 36 being pulled back by the first pull wire 34 while the second side 40 is not pulled back, to create a beveled tip (i.e., an adjustable tip 20 having a beveled angle). FIG. 3A depicts the second side 40 being pulled back by the second pull wire 38 while the first side 36 is not pulled back, to create a beveled tip. However, many other configurations are possible. For example, both of the first pull wire 34 and the second pull wire 38 may be pulled simultaneously, where one of the pull wires 34, 38 is pulled more than the other pull wire 34, 38, to still create an adjustable tip 20 having a beveled angle but a less pronounced beveled angle. The adjustable tip 20 may effectively have adjustable sections or segments created by the pull wires 34, 38 that can create any desired angle of tip. In certain embodiments, the adjustable tip 20 cannot be beveled by more than 45 degrees. However, this is not strictly necessary, and embodiments of the adjustable tip 20 that can be beveled by more than 45 degrees are encompassed within the scope of the present disclosure.

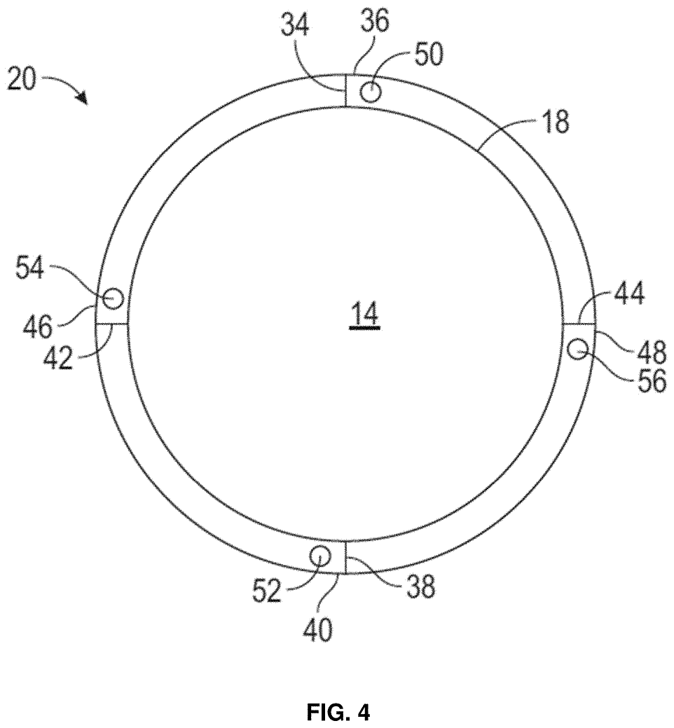

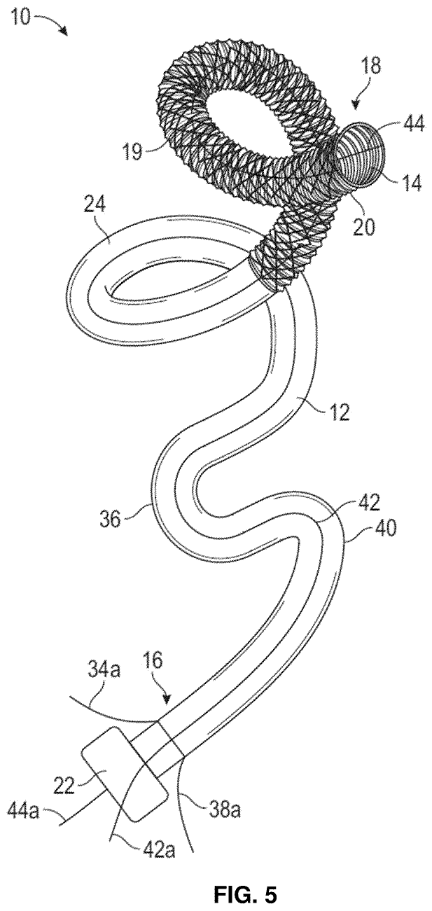

Although two pull wires 34, 38 are depicted in FIGS. 1-3 and described for example purposes, there may be more than two pull wires, and it is understood that additional pull wires may be attached, configured, and operated in the same manner as the pull wires 34, 38. In some embodiments, there are three pull wires, four pull wires, or five or more pull wires. In one non-limiting example, depicted in FIG. 4, the aspiration catheter 10 includes four pull wires 34, 38, 42, 44. Referring now to FIGS. 4-5, the aspiration catheter 10 may include a first pull wire 34 that extends and connects to a first side 36 of the adjustable tip 20, a second pull wire 38 that extends and connects to a second side 40 of the adjustable tip 20, a third pull wire 42 that extends along the tube 12 and connects to a third side 46 of the adjustable tip 20, and a fourth pull wire 44 that extends along the tube 12 and connects to a fourth side 48 of the adjustable tip 20. The third pull wire 42 may protrude from the proximal end 16 on the third side 46 to create a third pull wire protrusion 42a, and the fourth pull wire 44 may protrude from the proximal end 16 on the fourth side 48 to create a fourth pull wire protrusion 44a, where each of the third pull wire protrusion 42a and the fourth pull wire protrusion 44a functions in the same manner as the first pull wire protrusion 34a and the second pull wire protrusion 38a. That is, tension on the third pull wire protrusion 42a, or a force on the third pull wire protrusion 42a in a direction away from the distal end 18, such as from pulling on the third pull wire protrusion 42a, causes the third side 46 of the adjustable tip 20 to recede toward the proximal end 16. Tension on the fourth pull wire protrusion 44a, or a force on the fourth pull wire protrusion 44a in a direction away from the distal end 18, such as from pulling on the fourth pull wire protrusion 44a, causes the fourth side 48 of the adjustable tip 20 to recede toward the proximal end 16.

Referring still to FIGS. 4-5, the first side 36 is opposite the second side 40, and the third side 46 is opposite the fourth side 48. The first pull wire 34 terminates at a location on the adjustable tip located about 90 degrees around the circumference of the lumen 14 from where each of the third and fourth pull wires 42, 44 terminates on the adjustable tip 20. The second pull wire 38 also terminates at a location on the adjustable tip located about 90 degrees from where each of the third and fourth pull wires 44, 46 terminates on the adjustable tip 20. In other words, the four pull wires 34, 38, 42, 46 terminate at locations on the adjustable tip 20 that are effectively at the 12 o'clock, 3 o'clock, 6 o'clock, and 9 o'clock positions. However, the number of pull wires is not limited, and embodiments having more than four pull wires are encompassed within the scope of the present disclosure. Furthermore, the positioning of the pull wires 34, 38, 42, 46 on the adjustable tip 20 is not particularly limited; rather, any position and combination of pull wires that allows for the adjustable tip 20 to be adjusted in shape, angle, or configuration in a controlled manner is encompassed within the present disclosure.

Referring still to FIGS. 4-5, the aspiration catheter 10 may include one or more radiopaque markers to allow for visualization of the configuration of the adjustable tip 20 in use. For example, the adjustable tip 20 may include a first radiopaque marker 50 on the first side 36 near the first pull wire 34, a second radiopaque marker 52 on the second side 40 near the second pull wire 38, a third radiopaque marker 54 on the third side 46 near the third pull wire 42, and a fourth radiopaque marker 56 on the fourth side 48 near the fourth pull wire 44. The visualization from the radiopaque markers 50 ,52, 54, 56 allows the operator to see the configuration of the adjustable tip 20 and adjust it with the pull wires 34, 38, 42, 44 as needed in real time. It is understood that embodiments of the aspiration catheter 10 having fewer than four pull wires may still have radiopaque markers. For example, in the embodiment depicted in FIGS. 1-3, the aspiration catheter 10 may include a plurality of radiopaque markers, such as two or four radiopaque markers, to allow for visualization of the configuration of the adjustable tip 20 in use.

The aspiration catheter 10 may further include any of a number of features or elements known in the art for aspiration catheters, such as a microcatheter within the tube 12 to achieve a telescoping effect. The aspiration catheter 10 may include a microcatheter placed through the lumen 14 usable to deploy a retriever around a clot. The microcatheter and retriever can then be pulled back, with the goal of pulling the clot into the tube 12. The microcatheter may further or alternatively include expandable cages, baskets, or snares to capture and retrieve a clot. Furthermore, the adjustable tip 20 may be made extra rigid by incorporating two or more rings of nitinol. However, this is not strictly necessary.

Referring to FIG. 1, the aspiration catheter 10 can be used in conjunction with a suction device 17. A suction pump or other suction device 17 may be connected at the base 22 via a tube 21 to provide suction to the lumen 14 so as to be able to suck debris such as a thrombus into the lumen 14.

The aspiration catheter 10 may be inserted into a blood vessel directly or through a larger base catheter or sheath and guided to a location near a thrombus. The tube 12 is configured to be able to navigate to internal carotid artery, M1 or M2 segments of the middle cerebral artery, or other intracranial vessels such as the anterior or posterior cerebral arteries and the basilar artery. It can be a very tortuous, complex pathway to reach the brain. The operator may adjust the adjustable tip 20 with the pull wires 34, 38, 42, 44 while navigating through the tortuous blood vessels to the brain in order to adjust the shape and configuration of the adjustable tip 20 for easier passage to navigate side branches and to avoid vessel injuries. This provides great versatility for the operator using the aspiration catheter 10, and allows for intracranial vessels to be reached with less difficulty while minimizing the risk of puncturing or injuring other vessels in the process.

The operator may adjust the adjustable tip 20 with the pull wires 34, 38, 42, 44 and apply suction to the lumen 14 to suck the thrombus into the lumen 14 through the adjustable tip 20, thereby removing it from the blood vessel. The blood clots may be spongy, and can be, for example, 10-20 mm in length. Vigorous aspiration can be applied through the lumen 14 using the suction device 17 attached to the base 22 in order to remove the clots. The adjustment of the adjustable tip 20 while applying suction may allow for capturing any debris from the thrombus that breaks off during the process. The aspiration catheter 10 can thus be used for suctioning a clot out of a blood vessel in the brain. This can restore brain function early on as part of the standard of care for ischemic stroke. The aspirtation catheter 10 of the present disclosure allows for a more efficient and effective first pass to remove clots and restore brain function.

Certain embodiments of the device and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the device and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. An aspiration cathether comprising:
an elongated, flexible tube defining a lumen and extending from a proximal end to a distal end;
an adjustable tip at the distal end;
a first pull wire protruding from the proximal end to create a first pull wire protrusion and extending along the tube to the adjustable tip, wherein the first pull wire is connected to a first side of the adjustable tip; and
a second pull wire protruding from the proximal end to create a second pull wire protrusion and extending along the tube to the adjustable tip, wherein the second pull wire is connected to a second side of the adjustable tip;
wherein tension on the first pull wire causes the first side of the adjustable tip to recede toward the proximal end, and tension on the second pull wire causes the second side of the adjustable tip to recede toward the proximal end.

2. The aspiration catheter of claim 1, wherein the tension on the first pull wire is from pulling the fire pull wire protrusion, and the tension on the second pull wire is from pulling the second pull wire protrusion.

3. The aspiration catheter of claim 1, wherein the adjustable tip can be adjusted through the pull wires to form an angledor beveled tip.

4. The aspiration catheter of claim 1, wherein the adjustable tip comprises a first region of nitinol coil with a braid, and a second region of nitinoil coil with a braid, wherein the braid in the second region is less dense than the braid in the first region, and wherein the second region is closer to the distal end than the first region.

5. The aspiration catheter of claim 1, wherein the adjustable tip comprises a first region of nitinol coil with a braid, and a second region of nitinoil coil with no braid, wherein the second region is closer to the distal end than the first region.

6. The aspiration catheter of claim 1, wherein the elongated, flexible tube is at least 135 cm in length from the proximal end to the distal end.

7. The aspiration catheter of claim 1, further comprising at least one radiopaque marker on the adjustable tip.

8. The aspiration catheter of claim 1, further comprising two or more radiopaque markers on the adjustable tip.

9. The aspiration catheter of claim 1, wherein the aspiration catheter further comprises a third pull wire on a third side of the adjustable tip, and a fourth pull wire on a fourth side of the adjustable tip.

10. The aspiration catheter of claim 9, wherein the third pull wire protrudes from the proximal end to create a third pull wire protrusion and extends along the tube to the adjustable tip, and the fourth pull wire protrudes from the proximal end to create a fourth pull wire protrusion and extends along the tube to the adjustable tip;
wherein tension on the third pull wire from pulling the third pull wire protrusion causes the third side of the adjustbale tip to recede toward the proximal end, and tension on the fourth pull wire from pulling the fourth pull wire protrusion causes the fourth side of the adjustable tip to recede toward the proximal end.

11. The aspiration catheter of claim 9, further comprising a first radiopaque marker near the first pull wire, a second radiopaque marker near the second pull wire, a third radiopaque marker near the third pull wire, and a fourth radiopaque marker near the fourth pull wire.

12. The aspiration catheter of claim 1, wherein the elongated, flexible tube is fabricated from a memory alloy, a superelastic material, an elastic material, or a combination thereof.

13. The aspiration catheter of claim 1, wherein the elongated, flexible tube includes a hydrophilic coating on an outer surface thereof.

14. A method of removing a thrombus, the method comprising:
navigating the elongated, flexible tube of the aspiration catheter of claim 1 through a blood vessel to a thrombus;
applying tension on the first pull wire or the second pull wire to configure the adjustable tip in a manner desired for capturing the thrombus; and
applying suction to the elongated, flexible tube to remove the thrombus.

15. The method of claim 14, wherein the adjustable tip is configured to create a beveled tip.

16. The method of claim 15, further comprising releasing the tension on the first pull wire and/or second pull wire so as to configure the adjustable tip to create a non-beveled tip.

17. The method of claim 16, wherein the aspiration catheter further comprises a third pull wire and a fourth pull wire;
wherein the third pull wire protrudes from the proximal end to create a third pull wire protrusion and extends along the tube to the adjustable tip, and the fourth pull wire protrudes from the proximal end to create a fourth pull wire protrusion and extends along the tube to the adjustable tip; and
wherein tension on the third pull wire from pulling the third pull wire protrusion causes the third side of the adjustbale tip to recede toward the proximal end, and tension on the fourth pull wire from pulling the fourth pull wire protrusion causes the fourth side of the adjustable tip to recede toward the proximal end.

18. The method of claim 17, wherein the adjustable tip comprises two or more radiopaque markers configured to allow a user to visualize the manner in which the adjustable tip is configured.

19. The method of claim 18, wherein the adjustable tip comprises a first radiopaque marker on the first side, a second radiopaque marker on the second side, a third radiopaque marker on the third side, and a fourth radiopaque marker on the fourth side.

20. The method of claim 14, wherein the thrombus is located in a brain.
